(19) United States
(12) Patent Application Publication
(10) Pub. No.: US 2024/0407863 A1
(43) Pub. Date: Dec. 12, 2024

(54) ENDOLUMINAL ROBOTIC CATHETER CONTROLLER

(71) Applicant: Covidien LP, Mansfield, MA (US)

(72) Inventors: Matthew C. Blanco, Boston, MA (US); Jonathan Scott Thomson, Stoneham, MA (US); John M. Staton, Waltham, MA (US)

(73) Assignee: Covidien LP, Mansfield, MA (US)

(21) Appl. No.: 18/660,944

(22) Filed: May 10, 2024

Related U.S. Application Data
(60) Provisional application No. 63/472,129, filed on Jun. 9, 2023.

(51) Int. Cl. A61B 34/30; A61B 1/267; A61B 34/20

(52) U.S. Cl. CPC A61B 34/30; A61B 1/2676; A61B 34/20; A61B 2034/2051; A61B 2034/2074; A61B 2034/301; A61B 2034/303

(57) ABSTRACT

A control system, including a controller having a display and a handle detachably coupled to the display and including an input device, a motor assembly, wherein the display is detachably coupled to the motor assembly, the motor assembly including a drive motor in communication with the handle, and a catheter drive detachably coupled to the motor assembly, wherein actuation of the drive motor effectuates movement of a catheter operatively coupled to the catheter drive.

### CROSS-REFERENCE TO RELATED APPLICATION

This disclosure claims the benefit of, and priority to, U.S. Provisional Patent Application Ser. No. 63/472,129, filed on Jun. 9, 2023, the entire content of which is hereby incorporated by reference herein.

### BACKGROUND

### Technical Field

This disclosure relates to the field of medical device navigation, and in particular, to control systems for navigating a medical device to an area of interest.

### Description of Related Art

There are several commonly applied medical methods, such as endoscopic procedures or minimally invasive procedures, for treating various maladies affecting organs including the liver, brain, heart, lungs, gall bladder, kidneys, and bones. Often, one or more imaging modalities, such as magnetic resonance imaging (MRI), ultrasound imaging, computed tomography (CT), or fluoroscopy are employed by clinicians to identify and navigate to areas of interest within a patient and ultimately a target for biopsy or treatment. In some procedures, pre-operative scans may be utilized for target identification and intraoperative guidance. However, real-time imaging may be required to obtain a more accurate and current image of the target area. Furthermore, real-time image data displaying the current location of a medical device with respect to the target and its surroundings may needed to navigate the medical device to the target in a save and accurate manner (e.g., without causing damage to other organs or tissue).

For example, an endoscopic approach has proven useful in navigating to areas of interest within a patient, and particularly so for areas within luminal networks of the body such as the lungs, blood vessels, colorectal cavities, and the renal ducts. To enable the endoscopic approach, navigation systems have been developed that use previously acquired MRI data or CT image data to generate a three-dimensional (3D) rendering, model, or volume of the particular body part.

The resulting volume generated from the MRI scan or CT scan may be utilized to create a navigation plan to facilitate the advancement of a navigation catheter (or other suitable medical device) through a bronchoscope and a branch of the bronchus of a patient to an area of interest. A locating or tracking system, such as an electromagnetic (EM) tracking system, may be utilized in conjunction with, for example, CT data, to facilitate guidance of the navigation catheter through the branch of the bronchus to the area of interest. In certain instances, the navigation catheter may be positioned within one of the airways of the branched luminal networks adjacent to, or within, the area of interest to provide access for one or more medical instruments.

As will be appreciated, accurate placement of the catheter and therewith the medical instrument is important to ensure successful therapy. Improvements to the current navigation catheter systems are desired.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect of the disclosure is directed to a control system including a controller, the controller having: a display; and a handle detachably coupled to the display, the handle including an input device. The system also includes a motor assembly, where the display is detachably coupled to the motor assembly, the motor assembly including a drive motor in communication with the handle. The system also includes a catheter drive detachably coupled to the motor assembly, where actuation of the drive motor effectuates movement of a catheter operatively coupled to the catheter drive. Other embodiments of this aspect include corresponding computer systems, apparatus, and computer programs recorded on one or more computer storage devices, each configured to perform the actions of the methods.

Implementations may include one or more of the following features. The control system where the display includes a first coupler and the handle includes a second coupler, where the first coupler is selectively couplable to the second coupler. The first coupler includes a first magnet and the second coupler includes a second magnet. The first coupler includes a first plurality of magnets and the second coupler includes a second plurality of magnets, where the first plurality of magnets and the second plurality of magnets are disposed in a zig-zag configuration. The first coupler includes a first electrical connector and the second coupler includes a second electrical connector, where the first electrical connector is selectively engageable with the second electrical connector. The handle includes an energy storage device, where the first electrical connector and the second electrical connector cooperate to supply electrical energy to the energy storage device. The control system may include a docking station detachably coupled to the controller. The docking station includes a first plurality of magnets and the display of the controller includes a second plurality of magnets, where the first plurality of magnets and the second plurality of magnets cooperate to selectively couple the controller to the docking station. The insertion mechanism is operably coupled to the catheter to effectuate selective movement of the catheter. The motor assembly and the catheter drive cooperate to effectuate roll and articulation of the catheter and the insertion mechanism effectuates insertion and retraction of the catheter, or where the insertion mechanism includes a control knob, the control knob operably coupled to the catheter, where rotation of the control knob in a first direction effectuates advancement of the catheter and rotation of the control knob in a second direction effectuates retraction of the catheter. Implementations of the described techniques may include hardware, a method or process, or computer software on a computer-accessible medium.

A further general aspect of the disclosure is directed to a control station including a controller, the controller having: a housing, a first handle detachably coupled to the housing, a second handle detachably coupled to the housing. The station also includes a motor assembly detachably coupled to the controller, the motor assembly including a drive motor in communication with each of the first handle and the second handle. The station also includes a catheter drive detachably coupled to the motor assembly, where an input received by the first handle effectuates articulation of a catheter operatively connected to the catheter drive and an input received by the second handle effectuates linear movement of the catheter. Other embodiments of this aspect include corresponding computer systems, apparatus, and computer programs recorded on one or more computer storage devices, each configured to perform the actions of the methods.

Implementations may include one or more of the following features. The control station where the motor assembly includes: a first drive motor operably coupled to a first output hub; a second drive motor operably coupled to a second output hub; a third drive motor operably coupled to a third hub; and a fourth drive motor operably coupled to a fourth hub. The catheter drive includes; a first drive hub selectively engageable with the first output hub; a second drive hub selectively engageable with the second output hub; a third drive hub selectively engageable with the third output hub; and a fourth drive hub selectively engageable with the third output hub. Each of the first handle and the second handle are in wireless communication with the motor assembly. The motor assembly is in wireless communication with the housing. Implementations of the described techniques may include hardware, a method or process, or computer software on a computer-accessible medium.

A further aspect of the disclosure is directed to a controller including a housing, a display, and a first plurality of magnetic elements. The controller also includes a handle detachably coupled to the housing, the handle in wireless communication with the housing. The controller also includes a support including a second plurality of magnetic elements, where the second plurality of magnetic elements are selectively engageable with the first plurality of magnetic elements to selectively couple the housing to the support. Other embodiments of this aspect include corresponding computer systems, apparatus, and computer programs recorded on one or more computer storage devices, each configured to perform the actions of the methods.

Implementations may include one or more of the following features. The controller where the handle includes an energy storage device. The housing includes a first electrical connector and the handle includes a second electrical connector selectively engageable with the first electrical connector, the first electrical connector and the second electrical connector cooperating to transmit electrical energy from the housing to the energy storage device of the handle. The housing includes a first magnetic coupling and the handle includes a second magnetic coupling selectively engageable with the first magnetic coupling to selectively couple the handle to the housing. The first magnetic coupling includes a first plurality of magnetic elements disposed in a zig-zag configuration and the second magnetic coupling includes a second plurality of magnetic elements disposed in a complimentary configuration to the first plurality of magnetic elements. Implementations of the described techniques may include hardware, a method or process, or computer software on a computer-accessible medium.

### BRIEF DESCRIPTION OF THE DRAWINGS

Various aspects and embodiments of the disclosure are described hereinbelow with references to the drawings, wherein:

FIG. 1 is a schematic view of a navigation system in accordance with the disclosure;

FIG. 2 is a perspective view of a robotic system in accordance with aspects of the disclosure;

FIG. 3 is a front view of a control station for a robotic catheter system in accordance with the disclosure for use with the luminal network navigation system of FIG. 1;

FIG. 4 is a front, perspective view of the control station of FIG. 3 shown with a handle detached from a controller;

FIG. 5 is a rear view of the controller of FIG. 4;

FIG. 6 is a front, perspective view of a dock of the control station of FIG. 3;

FIG. 7 is a perspective view of the dock of FIG. 6 and a backplate of the controller of FIG. 4;

FIG. 8 is a bottom, perspective view of a motor assembly of the control station of FIG. 3;

FIG. 9 is a top, perspective view of a catheter drive of the control station of FIG. 3;

FIG. 10 is a front, perspective view of the controller of FIG. 4 shown with the motor assembly of FIG. 8 coupled to an upper portion of the controller;

FIG. 11 is a front, perspective view of another embodiment of a controller of the control system of FIG. 3 in accordance with the disclosure, shown with a single detachable handle and a motor assembly coupled to a right side of the controller;

FIG. 12 is front, perspective view of another embodiment of a control system in accordance with the disclosure, shown with a controller having two detachable handles coupled to an upper portion of the side surfaces of the controller;

FIG. 13 is a front, perspective view of the control system of FIG. 12, shown with the two detachable handles coupled to a lower portion of the side surfaces of the controller;

FIG. 14 is a front, perspective view of another embodiment of a control system in accordance with the disclosure, shown with two detachable handles coupled to an upper portion of the side surfaces of the controller;

FIG. 15 is a front, perspective view of the control system of FIG. 14, shown with the two detachable handles coupled to a lower portion of the side surfaces of the controller;

FIG. 16 is a perspective view of a catheter insertion mechanism of the control system of FIG. 3;

FIG. 17 is a perspective view of another embodiment of a catheter insertion mechanism of the control system of FIG. 3; and

FIG. 18 is a schematic view of a computing system in accordance with the disclosure.

### DETAILED DESCRIPTION

The disclosure is directed to a control station for facilitating navigation of an articulating catheter to an area of interest via a luminal network, such as the lungs. The control station includes a controller having a display and a pair of handles detachably coupled to the display. The handles include one or more input devices, such as a joystick, physical buttons, capacitive touch pads, etc. for control and navigation of a catheter through the luminal network. The handles may be detachably coupled to the display using magnets, buttons, or the like to enable a clinician to use the handles apart from the display. The handles are in communication with the display using a wireless or wired connection and may include accelerometers, inertial measurement units, etc. to detect and monitor movement of the handles.

The control station includes a motor assembly and a catheter drive, which is operably and detachably coupled to the motor assembly. The motor assembly includes one or more motors which are controlled via the handles, display, etc. The motor assembly includes a dock that is hingedly coupled to the motor assembly and enables the controller to be detachably supported on the dock via one or more magnets or the like. In this manner, a user may remove the controller from the motor assembly for remote operation. The dock enables a user to adjust an angle of the display via the hinged connection.

The catheter drive is detachably coupled to the motor assembly using magnets or the like. When coupled to the motor assembly, output hubs coupled to a driveshaft of each respective motor engage corresponding drive hub of the catheter drive, which in turn is engaged with a portion of the catheter shaft. In this manner, actuation of a motor effectuates rotation of the output hubs, which in turn effectuates rotation of the drive hubs to cause the catheter to advance, retract, roll, and/or articulate. As can be appreciated, operation of the drive motors is controlled by the handles, although control of the drive motors may be controlled concurrently with or separately from the handles.

In embodiments, the handles may be coupled to the display at a high position or a low position, depending on the preferences of the user. It is envisioned that the controller may not have a display, and rather, the handles may be coupled to a dummy controller or panel and the user may view a separate display. It is contemplated that the motor assembly may be disposed in any orientation, such that when the controller is coupled to the dock, the motor assembly may be disposed above the display, below the display, to the right of the display, etc.

The control station may be operably coupled to a catheter insertion mechanism that is disposed adjacent a body cavity of the patient, such as the patient's mouth. The catheter insertion mechanism includes one or more motors that are operably coupled to one or more drive gears or wheels. The drive gears abut or otherwise contact a portion of the catheter shaft, such that actuation of the motor effectuates advancement or retraction of the catheter. In embodiments, the catheter insertion mechanism may include a manual control knob, which when manipulated by the user, manually advances or retracts the catheter. These and other aspects of the disclosure will be described in further detail hereinbelow.

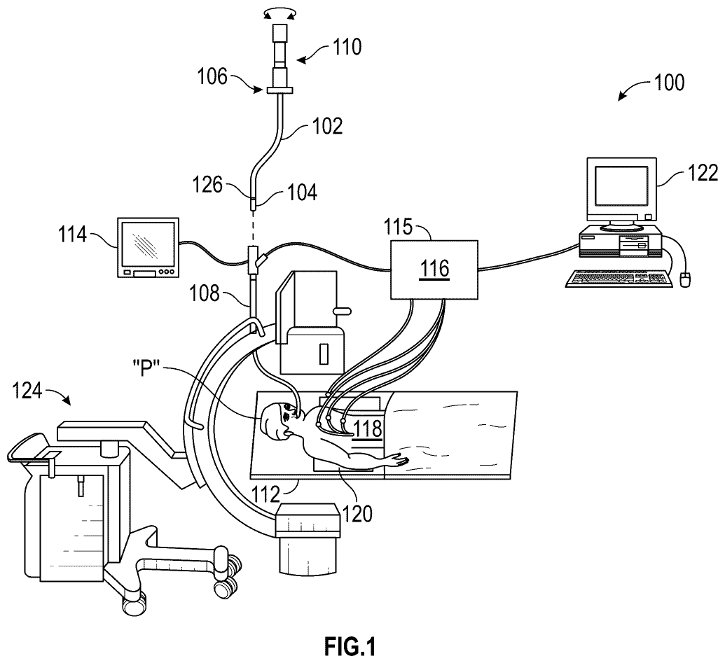

With reference to FIG. 1, a navigation system facilitating navigation of a medical device through a luminal network and to an area of interest is illustrated and generally identified by reference numeral 100. The navigation system 100 includes a catheter 102 and a guide assembly 106. In one embodiment, the catheter 102 is inserted into a bronchoscope 108 for access to a luminal network of the patient P. In this manner, the catheter 102 may be inserted into a working channel of the bronchoscope 108 for navigation through a patient's luminal network, such as the lungs. The catheter 102 may itself include imaging capabilities via an integrated camera or optics component 109 and therefore, a separate bronchoscope 108 is not strictly required. A locatable guide (LG) 110 (a second catheter), including a sensor 104 may be inserted into the catheter 102 and locked into position such that the sensor 104 extends a desired distance beyond a distal tip of the catheter 102. As can be appreciated, the position and orientation of the sensor 104 relative to a reference coordinate system, and thus the distal portion of the catheter 102, within an electromagnetic field can be derived. Catheter guide assemblies 106 are currently marketed and sold by Medtronic PLC under the brand names SUPERDIMENSION® Procedure Kits, or EDGE® Procedure Kits, and are contemplated as being usable with the disclosure.

Continuing with FIG. 1, the system 100 generally includes an operating table 112 configured to support a patient P and monitoring equipment 114 coupled to the bronchoscope 108 or catheter 102 (e.g., a video display, for displaying the video images received form the video imaging system of the bronchoscope 108 or the catheter 102), a locating or tracking system 115 including a tracking module 116, a plurality of reference sensors 118 and a transmitter mat 120 including a plurality of incorporated markers, and a computing device 122 including software and/or hardware used to facilitate identification of a target, pathway planning to the target, navigation of a medical device to the target, and/or confirmation and/or determination of placement of the catheter 102, or other suitable device therethrough, relative to the target.

A six degrees-of-freedom electromagnetic locating or tracking system 115, or other suitable system for determining position and orientation of a distal portion of the catheter 102, is utilized with the LG 110, as will be described in further detail hereinbelow, for performing registration of a detected position of the sensor 104 and a 3D model generated from a CT or MRI image scan. The tracking system 115 is configured for use with the LG 110 and particularly with the sensor 104. As described hereinabove, the LG 110 and the sensor 104 are configured for insertion through the catheter 102 into the patient P's airways (either with or without the bronchoscope 108) and are selectively lockable relative to one another via a locking mechanism (not shown).

With continued reference to FIG. 1, the transmitter mat 120 is positioned beneath the patient P. The transmitter mat 120 generates an electromagnetic field around at least a portion of the patient P within which the position of the plurality of reference sensors 118 and the sensor 104 can be determined with the use of the tracking module 116. A second electromagnetic sensor 126 may also be incorporated into the end of the catheter 102. The second electromagnetic sensor 126 may be a five degree-of-freedom sensor or a six degree-of-freedom sensor. One or more of the reference sensors 118 are attached to the chest of the patient P. Registration is generally performed to coordinate locations of the 3D model and 2D images from the planning phase, with the patient P's airways as observed through the bronchoscope 108 and allow for the navigation phase to be undertaken with knowledge of the location of the sensor 104.

Registration of the patient P's location on the transmitter mat 120 may be performed by moving the sensor 104 through the airways of the patient P. In this manner, data pertaining to locations of the sensor 104, while the LG 110 is moving through the airways, is recorded using the transmitter mat 120, the references sensors 118, and the tracking system 115. A shape resulting from this location data is compared to an interior geometry of passages of a 3D model, and a location correlation between the shape and the 3D model based on the comparison is determined, e.g., utilizing the software on the computing device 122. In addition, the software identifies non-tissue space (e.g., air filled cavities) in the 3D model. The software aligns, or registers, an image representing a location of the sensor 104 with the 3D model and/or 2D images generate from the 3D model, which are based on the recorded location data and an assumption that the LG 110 remains located in non-tissue space in patient P's airways. Alternatively, a manual registration technique may be employed by navigating the bronchoscope 108 with the sensor 104 to pre-specified locations in the lungs of the patient P, and manually correlating the images from the bronchoscope 108 to the model data of the 3D model. Although generally described with respect to EMN systems using EM systems, the instant disclosure is not so limited and may be used in conjunction with flexible sensors, such as fiber-bragg grating sensors, inertial measurement units (IMU), ultrasonic sensors, without sensors, or combinations thereof.

In accordance with aspects of the disclosure, the visualization of intra-body navigation of a medical device (e.g., a biopsy tool or a therapy tool), towards a target (e.g., a lesion) may be a portion of a larger workflow of a navigation system. An imaging device 124 (e.g., a CT imaging device such as a cone-beam computed tomography (CBCT) device, including but not limited to Medtronic plc's O-arm™ system) capable of acquiring 2D and 3D images or video of the patient P is also included in the particular aspect of system 100. The images, sequence of images, or video captured by imaging device 124 may be stored within the imaging device 124 or transmitted to the computing device 122 for storage, processing, and display. In embodiments, the imaging device 124 may move relative to the patient P so that images may be acquired from different angles or perspectives relative to the patient P to create a sequence of images, such as a fluoroscopic video. The pose of the imaging device 124 relative to the patient P while capturing the images may be estimated via markers incorporated with the transmitter mat 120. The markers are positioned under the patient P, between the patient P and the operating table 112, and between the patient P and a radiation source or a sensing unit of the imaging device 124. The markers incorporated with the transmitter mat 120 may be two separate elements which may be coupled in a fixed manner or alternatively may be manufactured as a single unit. It is contemplated that the imaging device 124 may include a single imaging device or more than one imaging device.

Continuing with FIG. 1, the computing device 122 may be any suitable computing device including a processor and a storage medium, wherein the processor is capable of executing instructions stored on the storage medium. The computing device 122 may further include a database configured to store patient data, CT data sets including CT images, fluoroscopic data sets including images and video, 3D reconstructions, navigation plans, and any other such data. Although not explicitly illustrated, the computing device 122 may include inputs, or may otherwise be configured to receive CT data sets, fluoroscopic images/video, and other data described herein. The computing device 122 includes a display configured to display graphical user interfaces. In embodiments, the computing device 122 may be connected to one or more networks through which one or more databases may be stored.

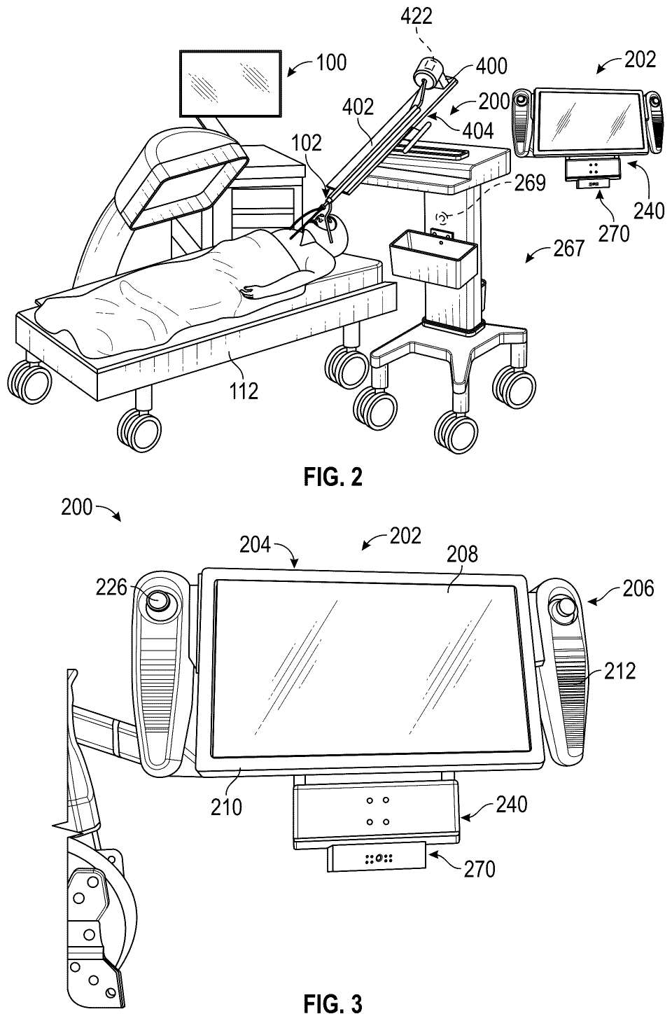

Turning to FIGS. 2 and 3, a control station for use with the system 100 is illustrated and generally identified by reference numeral 200. The control station 200 is a robotic surgical system, although in embodiments, it is envisioned that the control station 200 may be a standalone system that is configured to communicate with existing robotic surgical systems without departing from the scope of the disclosure. The control station 200 includes a controller 202, a motor assembly 240, and a catheter drive 270. The controller 202 includes a display 204 and a pair of handles 206 operably coupled to the display 204. The display 204 includes a display screen 208 and a housing or casing 210 operably coupled to the display 204. It is contemplated that the housing 210 may be integral with the display screen 208 or may be a separate component from the display screen 208 such that the housing 210 encapsulates a portion of the display screen 208. It is contemplated that the display screen 208 may be any suitable display capable of displaying images, video, user interfaces, etc., and in one non-limiting embodiment is a tablet or other suitable portable computing device. The display 204 communicates with the system 100, such as the computing device 122, the tracking system 115, the endoscope 104 or the bronchoscope 108, etc. via a wired or wireless connection and may include one or more communication ports, amongst others. In embodiments, the display screen 208 may only display images, videos, user interfaces, etc. and computing operations may be handled externally, such as by the computing device 122.

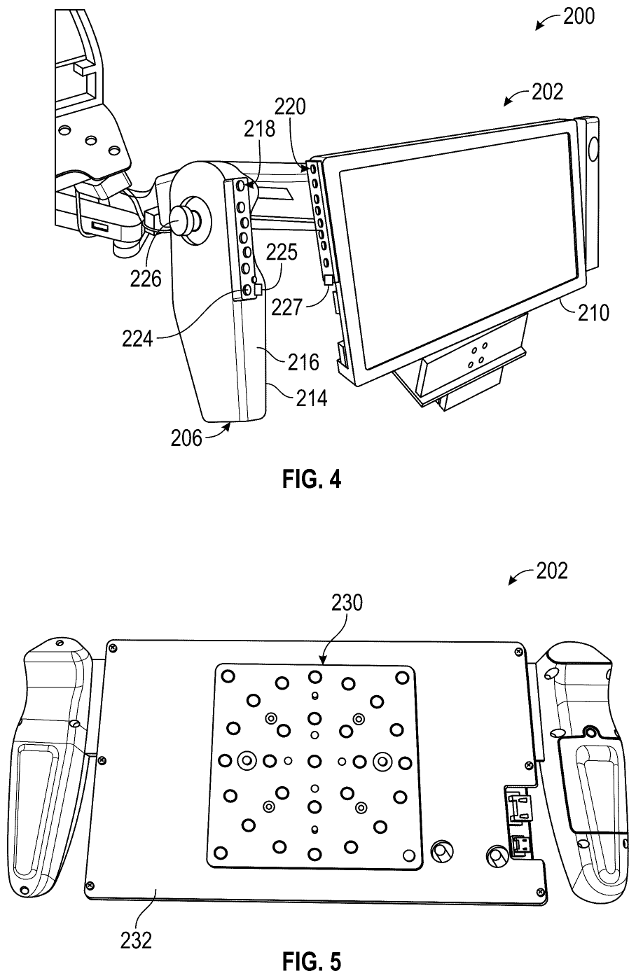

With additional reference to FIG. 4, each handle of the pair of handles 206 is substantially similar to one another and therefore, only one handle 206 will be described in detail herein in the interest of brevity. The handle 206 defines a generally oblong or elliptical profile having an upper surface 212, an opposite lower surface 214, and a side surface 216 extending between each of the upper surface 212 and lower surface 214. A coupler 218 is disposed on a portion of the side surface 216 of the handle that interfaces with a corresponding coupler 220 disposed on a side surface of the housing 210 of the display 204. As can be appreciated, the coupler 218 facilitates selective attachment and detachment of the handle 206 from the display 204. Although generally illustrated as utilizing one or more magnets 224, it is contemplated that the coupler 218 may use any suitable means to selectively couple the handle 206 to the housing 210, such as hook and loop, twist couplings (e.g., quarter turn, half turn, etc.), snap fasteners (e.g., buttons, etc.), snap joints (e.g., fingers and slots) amongst others. It is envisioned that the magnets 224 may be disposed in a zig-zag or sinusoidal configuration along a length of the coupler 218 to provide stability and help align the handle 206 with the housing 210 of the display 204, although it is contemplated that any suitable configuration may be utilized without departing from the scope of the present disclosure. The handle 206 may include one or more locating features, such a pin and bore arrangement to facilitate alignment of the handle 206 relative to the display 204 and inhibit twisting or other undesirable movement of the handle 206 relative to the display 204. In embodiments, the couplers 218 and 220 may include one or more electrical connectors 225 and 227, respectively, that engage with one another when the handle 206 is coupled to the display 204 and disengage from one another when the handle 206 is removed from the display 204. As can be appreciated, the electrical connectors 225 and 227 may facilitate charging of an energy storage device (e.g., a battery) disposed within a portion of the handle 206 or any other suitable feature, such as communication, etc.

The handle 206 includes an input device, such as for example, a joystick or a control handle 226 extending from or through the upper surface 212. The joystick 226 is configured to be grasped or otherwise manipulated by a user. It is envisioned that the joystick 226 may be manipulated 360 degrees about its axis, although it is contemplated that the joystick 226 may include detents or other mechanical features to limit the joystick 226 to any suitable number of positions, such as two (e.g., up and down or left and right), four, etc. The handle 206 is operably coupled to the display 204 via a wireless connection, such as Bluetooth, Wifi, etc. to enable inputs received by the joystick 226 to be communicated to the display 204, although a wired connection between the handle 206 and the display 204 is contemplated. In embodiments, in addition to the joystick 226, the handle 206 may include one or more input receiving devices (not shown), such as physical buttons, touch-sensitive pads, scroll wheels, etc., haptic feedback devices, and sensors (e.g., IMU's, accelerometers, etc.) for detecting movement or gestures made by the user when grasping the handle 206. In embodiments, the joystick 226 may be depressible (e.g., actuatable in a direction towards the lower surface 214) to act as a button or other suitable device. In addition to, or in lieu of the joystick 226, it is envisioned that the handle 206 may include a control pad (e.g., directional pad, touch sensitive pad, etc.).

The handle 206 controls the operation of the catheter 104 (or in embodiments, the bronchoscope 108), such as steering, insertion, retraction, etc. It is envisioned that each handle of the pair of handles 206 may control separate operations of the catheter, such as a first handle 206 controlling steering and a second handle 206 controlling insertion and retraction. In embodiments, the second handle 206 may act as a redundant steering handle. Although generally described as having a pair of handles 206, it is envisioned that the controller 202 may include only one handle 206, which is operable to control only steering of the catheter 104, although it is envisioned that the single handle 206 may control any aspect of the catheter 104 without departing from the scope of the present disclosure.

It is envisioned that one or both of the pair of handles 206 may include audio and/or visual devices (not shown) configured to provide various alerts or other information to the user. In this manner, the handle 206 may include an audio device (e.g., a speaker, buzzer, etc.) to emit an alarm, a note or notes indicating connectivity status with the display 204 (e.g., a chime indicating that the handle 206 is wirelessly connected to the display 204, the handle 206 is unable to connect to the display 204, the handle is in the process of connecting to the display, etc.). It is contemplated that the audio device may alert the user to navigation states during the procedure being performed or the like. In embodiments, the visual device may be utilized in lieu or in combination with the audio device, and may alert or otherwise indicate to the user various information regarding the connectivity status of the handle 206, a navigation status during the procedure being performed, etc. In one non-limiting embodiment, the handle 206 may include one or more paging buttons having an audio/visual indication when actuated (e.g., a chime when depressed or released, a colored LED when depressed or released, etc.). As can be appreciated, the handle 206 may include any audio/visual devices or systems for indicating any suitable alert, alarm, status, etc. without departing from the scope of the present disclosure.

With reference to FIGS. 5-7, the controller 202 includes a docking station or support 228 to detachably support and couple to a corresponding coupling 230 disposed on a rear surface 232 of the display 204. As can be appreciated, the coupler 230 of the display 204 includes corresponding features to the support 228, and therefore, will not be described in detail herein in the interest of brevity. The support 228 defines a generally square profile, although it is contemplated that the support 228 may define any suitable profile, such as rectangular, circular, oval, amongst others. One or more coupling devices 233 are disposed or formed on a front surface 234 of the support 228 that are configured to selectively engage a corresponding coupling device or devices of the display 204, as will be described in further detail hereinbelow. Although generally illustrated as being a plurality of magnets, it is contemplated that the coupling devices 233 may be any suitable device capable of selectively coupling the display 204 to the support 228, such as hook and loop, twist couplings (e.g., quarter turn, half turn, etc.), snap fasteners (e.g., buttons, etc.), snap joints (e.g., fingers and slots), amongst others. It is envisioned that the coupling devices 233 may be arranged in any suitable fashion, and in one non-limiting embodiment, are arranged in a plurality of concentric rings. In embodiments, the support 228 may include one or more locating features, such as a pin and bore arrangement, to facilitate alignment of the display 204 relative to the support 228. It is envisioned that the support 228 may include one or more electrical connectors (not shown) that engage with one another when the display 204 is coupled to the support 228 and disengage from one another when the display 204 is removed from the support 228. It is contemplated that the electrical connectors may facilitate charging of an energy storage device (e.g., a battery) disposed within a portion of the display 204 or any other suitable feature, such as communication, etc. The support 228 is pivotably coupled to a portion of the motor assembly 240 using any suitable means, such as a hinge 235 (which, in embodiments may be a living hinge). It is envisioned that the hinge 235 may include one or more detents, stops, locks, etc. to permit selective adjustment of an angle of the support 228 relative to the motor assembly 240 and lock or otherwise maintain the desired position of the support 228 relative to the motor assembly 240.

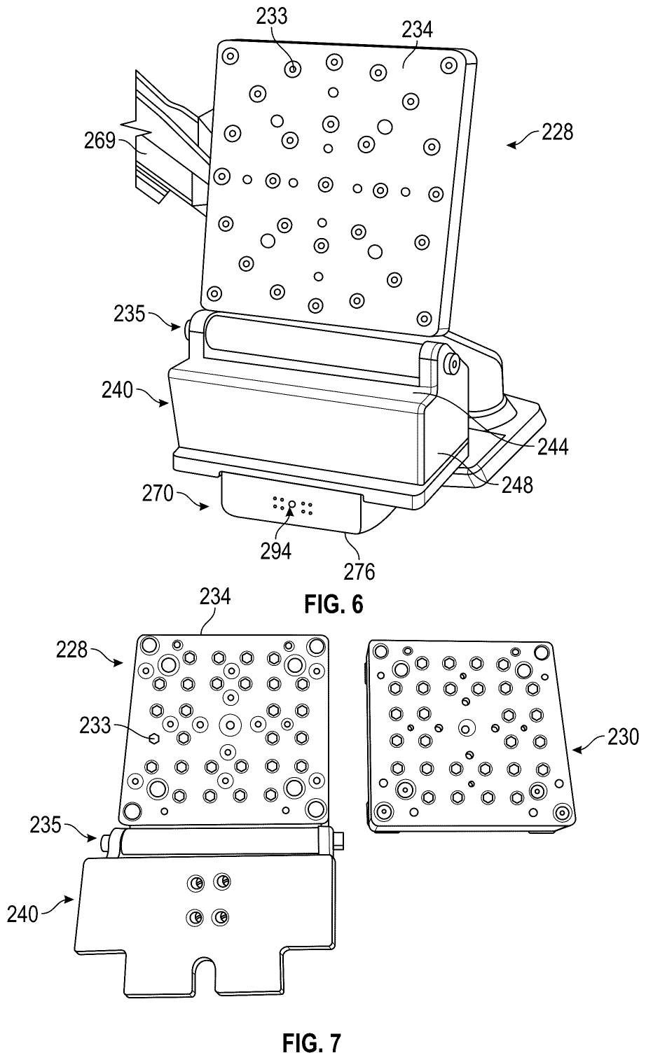

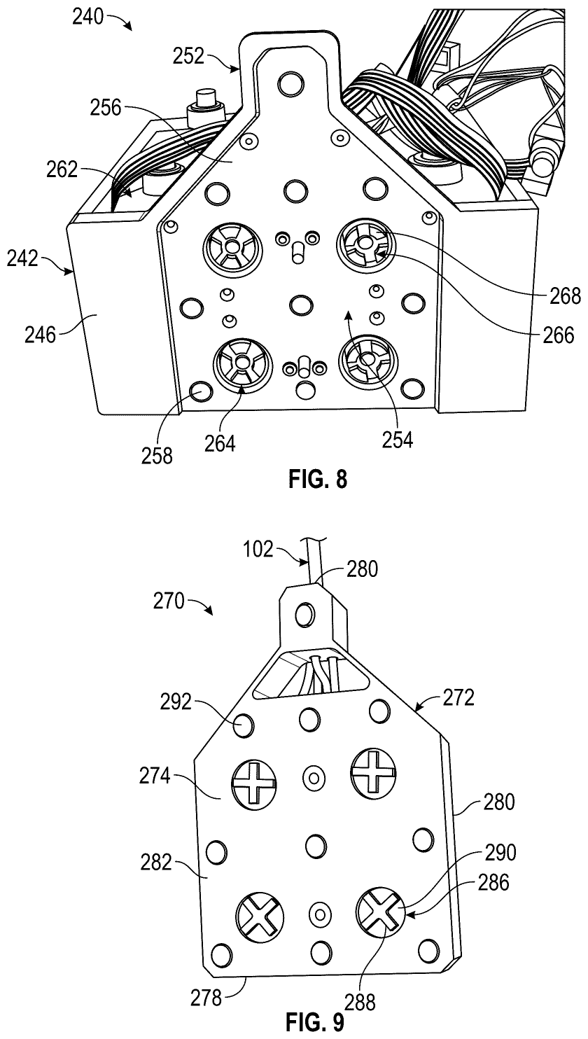

With reference to FIGS. 6, 8, and 9, the motor assembly 240 includes a housing 242 having a top surface 244, an opposed bottom surface 246 (FIG. 8), and an outer surface 248 extending between and around a perimeter of the top and bottom surfaces 244, 246. The top surface 244 of the housing 242 is operably coupled to the support 228 in a manner described in further detail herein. The bottom surface 246 of the housing 242 includes a protuberance 252 extending past one side of the outer surface 248. A relief or counterbore 254 is defined within the bottom surface 246 for selective receipt of a complimentary shaped portion of the catheter drive 270, as will be described in further detail hereinbelow. Although generally illustrated as defining a profile that is substantially similar to the protuberance 242, it is envisioned that the relief 254 may define any suitable profile without departing from the scope of the present disclosure. The relief 254 defines a generally planar surface 256. One or more coupling devices 258 are disposed on or recessed within the planar surface 256 of the relief 254 for selectively coupling the catheter drive 270 to the housing 242. Although generally illustrated as being one or more magnets, it is contemplated that the coupling device 258 may employ any suitable means to selectively couple the catheter drive 270 to the housing 242, such as hook and loop, twist couplings (e.g., quarter turn, half turn, etc.), snap fasteners (e.g., buttons, etc.), snap joints (e.g., fingers and slots) amongst others. It is envisioned that the magnets 258 may be disposed in any suitable arrangement, and in one non-limiting embodiment, are arranged in a row and column manner. The planar surface 256 of the relief 254 may include one or more locating features, such a pin and bore arrangement to facilitate alignment of the catheter drive 270 relative to the housing 242 of the motor assembly 240 and inhibit twisting or other undesirable movement of the catheter drive 270 relative to the housing 242. In embodiments, the planar surface 256 of the relief 254 and a portion of the catheter drive 270 may include one or more electrical connectors (not shown) that engage with one another when the catheter drive 270 is coupled to the housing 242 and disengage from one another when the catheter drive 270 is removed from the housing 242.

The housing 242 defines a hollow interior portion 262 in which one or more drive motors (not shown) are disposed. In one non-limiting embodiment, four drive motors are disposed within, and supported on the hollow inner portion 262. Each drive motor includes an output hub 264 having a relief 266 defined within an outer surface 268. Although generally illustrated as having a “cross” or “plus” shaped configuration, it is envisioned that the relief 266 may include any suitable profile capable of transmitting rotation of the motor to a corresponding feature of the catheter drive 270, as will be described in further detail hereinbelow. The housing 242 is operably coupled to and supported on a workstation or adjustable arm 267 (FIGS. 2 and 6). As can be appreciated, the housing 242 anchors or otherwise supports the controller 202 on the arm 267. It is envisioned that the housing 242 may be coupled to the arm 269 using any suitable means, and may be selectively or fixedly coupled to the arm depending upon the design needs of the control station 200. In embodiments, the workstation or adjustable arm 267 may include a wireless or wired communication system 269 to facilitate communication between the controller 202 and the system 100, such as for example, the computing device 122.

The drive motors are in communication with the controller 202 or computing device 122, such that inputs received by the controller 202 are communicated to the drive motors to effectuate movement and navigation of the catheter 102. In embodiments, the four drive motors may be associated with a corresponding motion, such as roll, articulation, advancing, retracting, etc., although it is contemplated that one or more of the four drive motors may cooperate to effectuate motion of the catheter 102. It is envisioned that the drive motors may be operably coupled to the controller 202 or the computing device 122 using any suitable means, such as a wired connection or a wireless connection.

Continuing with FIGS. 6, 8, and 9, the catheter drive 270 includes a housing 272 defining an upper surface 274, an opposed lower surface 276 (FIG. 6), a first end surface 278 and a second, opposite end surface 280 extending between opposed first and second side surfaces 282 and 284 and the upper and lower surface 274, 276. The housing 272 defines a hollow interior portion (not shown) for receipt of one or more gears or drive wheels (not shown) for engaging with, and effectuating movement of the catheter 102. As can be appreciated, the number of gears corresponds to the number of drive motors of the motor assembly 240, although it is contemplated that the catheter drive 270 may include any number of gears, which may be the same or different than the number of drive motors of the motor assembly 240. In embodiments, the drive gears may be one or more gear trains depending upon the design needs of the control station 200. Each drive gear is operably coupled to a corresponding drive hub 286 protruding through the upper surface 274. Each drive hub 286 includes a protuberance 288 disposed on and extending from an outer surface 290. As can be appreciated, the protuberance 288 includes a profile that is generally complimentary to the profile of the relief 266 of the output hubs 264 such that rotation of the output hubs 264 effectuates a corresponding rotation of the drive hub 286.

One or more coupling devices 292 are disposed on or recessed within the upper surface 274 that are configured to selectively engage a corresponding coupling device 258 of the housing 242 of the motor assembly 240. In this manner, the coupling devices 258 and 292 selectively couple the catheter drive 270 to the motor assembly 240, and therefore, cause the protuberances 288 of the drive hubs 286 to be received within the corresponding reliefs 266 of the output hubs 264 to couple the drive motors of the drive assembly 240 to the gears of the catheter drive 270. The outer profile of the housing 272 of the catheter drive 270 is generally complimentary to the profile of the relief 254 of the housing 242 of the drive assembly 240 such that the housing 272 of the catheter drive is received within the relief 254 to inhibit or otherwise prevent movement of the catheter drive 240 relative to the drive assembly 240.

A throughbore 294 is defined through each of the first and second end surfaces 278, 280 for receipt of a portion of the catheter 102 or a tool. As can be appreciated, the throughbore 294 is interposed between pairs of gears (arranged in a square arrangement). In this manner, when the catheter 102 is inserted within and through the throughbore 294, a portion of the catheter 102 engages or otherwise contacts a portion of a respective gear of the catheter drive 240 to effectuate movement of the catheter 102. In embodiments, the catheter 102 may be selectively coupled to a portion of the throughbore 294 and a portion of the tool (not shown) is exposed to and engages with the gears to enable control of the tool separate from the catheter 102.

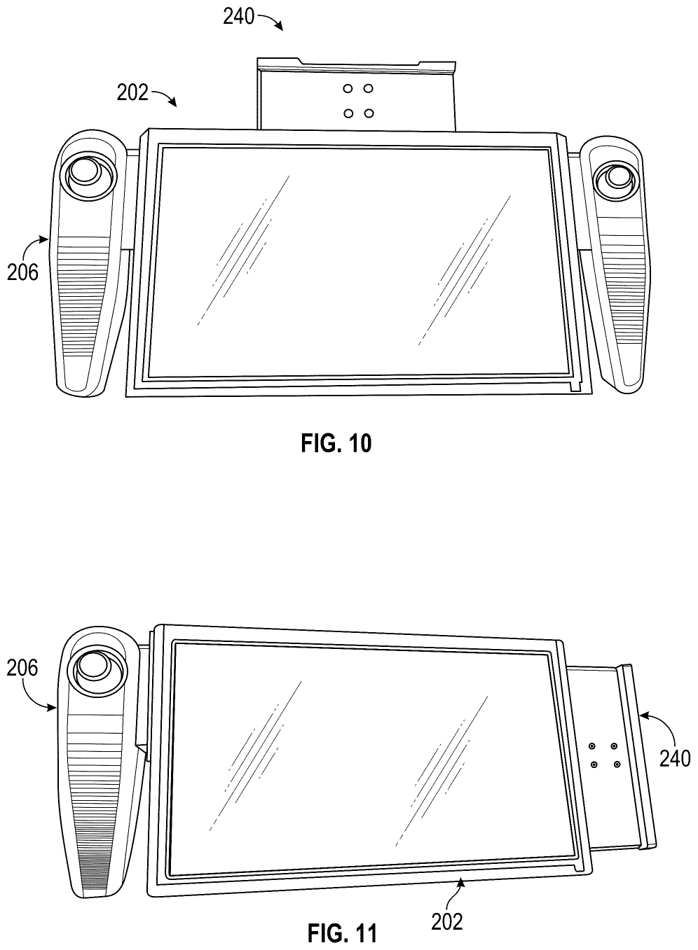

Although generally illustrated as being oriented at a lower portion of the controller 202, it is envisioned that the motor assembly 240 may be oriented in any suitable direction, such as at the upper portion of the controller 202 (FIG. 10) or to the left (FIG. 11) or right of the controller 202, etc. In embodiments, where the motor assembly 240 is oriented to the left or right of the controller 202, the controller 202 may include only one handle 206 (FIG. 11) or may include two handles 206.

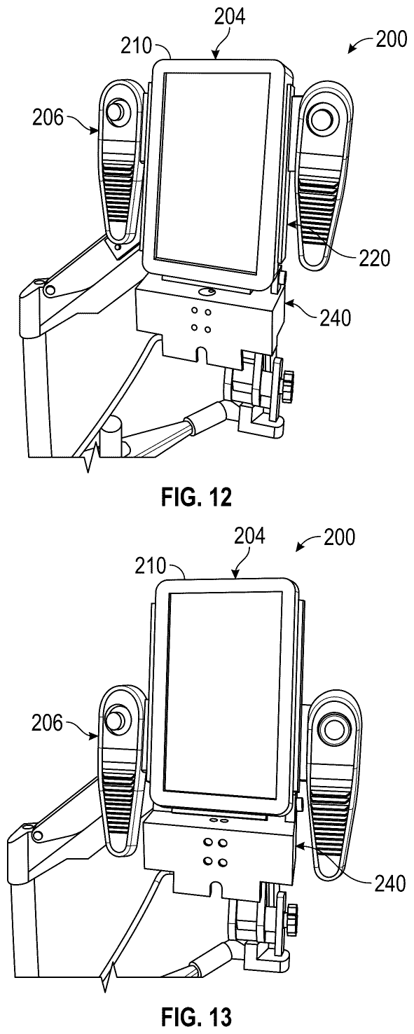

With reference to FIGS. 12 and 13, it is envisioned that the display 204 may be oriented in a vertical direction in lieu of a horizontal direction. In embodiments, one or both pair of handles 206 may be disposed in a high position (e.g., away from the motor assembly 240; FIG. 12)) or may be disposed in a low position (e.g., towards the motor assembly 240; FIG. 13). It is envisioned that the housing 210 of the display 204 may include two pairs of couplers 220, with each pair of couplers 220 disposed at a high position and a low position to enable the user to couple the pair of handles 206 in either location as desired.

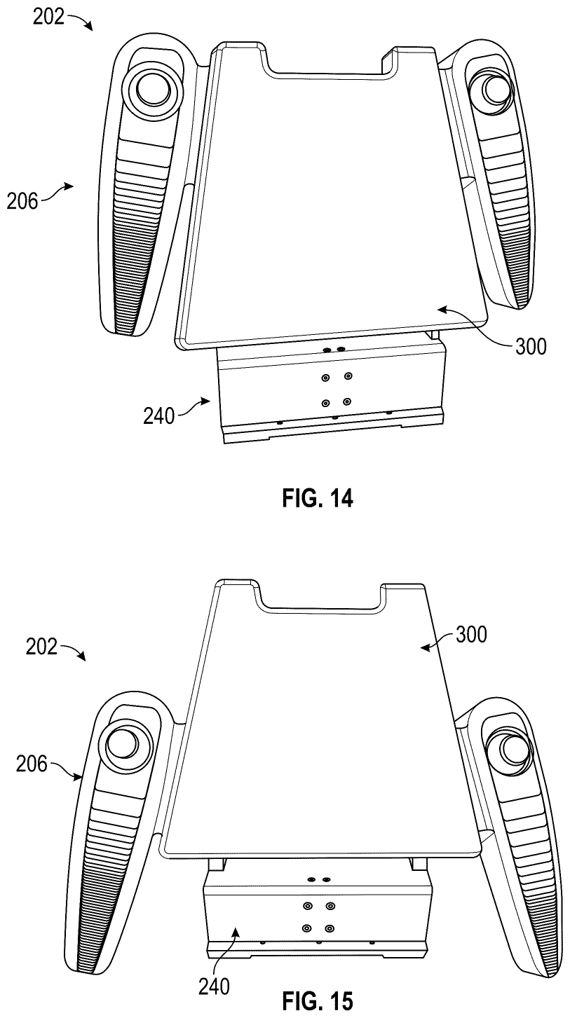

With reference to FIGS. 14 and 15, it is envisioned that the controller 202 may not include a display 204, and rather, the user may use a display associated with the computing device 122 or the monitoring equipment 114. In this manner, the pair of handles 206 are operably coupled to a dummy controller or panel 300 using any of the coupling methods described herein. It is envisioned that the pair of handles 206 may be coupled to the panel 300 in a high position (FIG. 14) or a low position (FIG. 15) to accommodate preferences of the user.

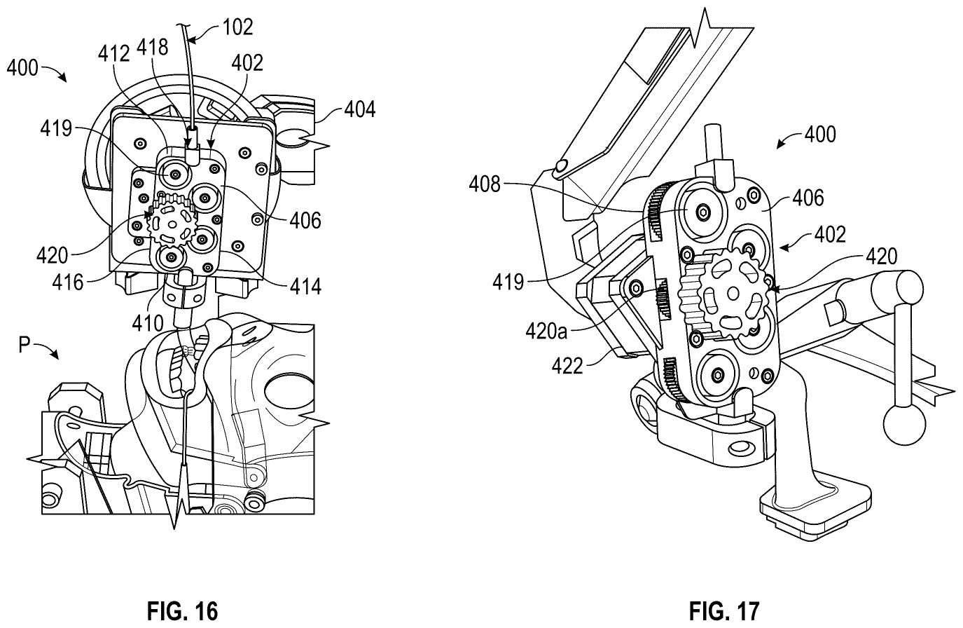

Turning to FIGS. 16 and 17, a catheter insertion mechanism is illustrated and generally identified by reference numeral 400. The catheter insertion mechanism 400 includes a drive housing 402 operably coupled to a support arm 404 that is different from the adjustable arm 269 that supports the control station 200, although it is envisioned that both the drive housing 402 of the catheter insertion mechanism 400 and the control station 200 may be supported on the same support arm. It is envisioned that the support arm 404 may be stationary, manually adjustable, remotely adjustable, or combinations thereof. The housing 402 defines a front surface 406, an opposite rear surface 408 and a lower surface 410 and a opposite upper surface 412 extending between the front and rear surfaces 406, 408 and opposed first and second side surfaces 414 and 416. A bore 418 is defined through the lower surface 410 and the upper surface 412 that is configured to receive a portion of the catheter 102. In this manner, the catheter 102 is permitted to pass entirely through the bore 418 and into a body cavity of the patient, which may be patient's mouth.

The housing 402 defines a hollow interior portion (not shown) in which one or more drive gears or drive wheels 419 are rotatably disposed. Although generally illustrated as protruding through the front surface 406, it is envisioned that the drive gears 419 may be entirely encapsulated within the housing 402. In one non-limiting embodiment, the catheter insertion mechanism 400 includes four drive gears 419, with two drive gears 419 disposed on a first side of the bore 418 (e.g., towards the first side surface 414) and the remaining two drive gears 419 disposed on a second, opposite side of the bore 418 (e.g., towards the second side surface 416). Although generally described as having four drive gears 419, it is envisioned that the catheter insertion mechanism 400 may include any suitable number of drive gears 419 that may be arranged in any suitable orientation without departing from the scope of the present disclosure. The drive gears 419 are disposed within the housing 402 of the catheter insertion instrument 400 at a location where a portion of each drive gear 419 abuts or otherwise contacts a portion of the catheter 102. In this manner, rotation of the drive gears 419 effectuates a corresponding movement of the catheter 102 to advance or retract the catheter 102. The housing 402 includes a manual control gear or wheel 420a (FIG. 17) rotatably supported within the cavity that is in mechanical communication with the catheter 102, or in embodiments, one or more of the drive gears 419. A manual control knob 420 is operably coupled to the manual control gear to enable a user to manually advance or retract the catheter 102 received within the bore 418. The catheter insertion mechanism 400 includes one or more insertion motors 422 supported on or operably coupled to the housing 402. The motor 422 is operably coupled to one or more of the drive gears 419. In this manner, actuation of the motor 422 in a first direction effectuates advancement of the catheter 102 through the bore 418 and actuation of the motor 422 in a second, opposite direction effectuates retraction of the catheter 102 through the bore 418.

It is envisioned that the catheter insertion mechanism 400 may be utilized in conjunction with or separate from the motor assembly 240 and catheter drive 270. In this manner, the motor assembly 240 and catheter drive 270 may effectuate roll and articulation of the catheter 102 whereas the catheter insertion mechanism 400 may effectuate insertion and retraction of the catheter 102. In embodiments, the catheter insertion mechanism 400 may be utilized as a redundant insertion and retraction device. It is contemplated that the catheter insertion mechanism 400 may be in wireless or wired connection with one or both of the control station 200 or computing device 122 using any suitable method.

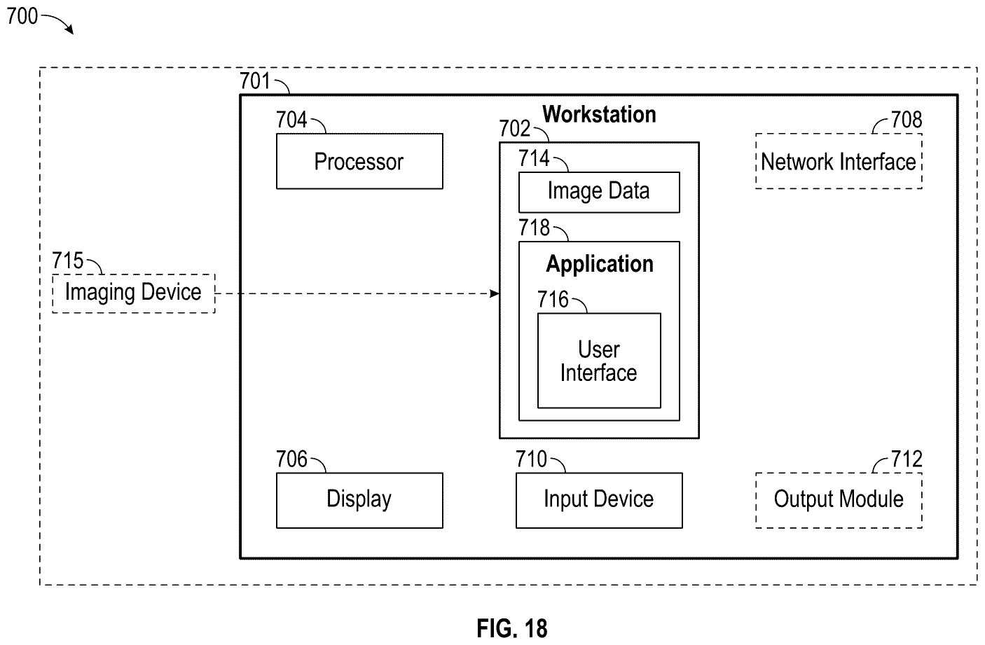

Reference is now made to FIG. 18, which is a schematic diagram of a system 700 configured for use with the devices and systems of the disclosure. System 700 may include a workstation 701, and optionally an imaging device 715 (e.g., a fluoroscope, ultrasound device, or CBCT device). In some embodiments, workstation 701 may be coupled with imaging device 715, directly or indirectly, e.g., by wireless communication. Workstation 701 may include a memory 702, a processor 704, a display 706 and an input device 710. Processor or hardware processor 704 may include one or more hardware processors. Workstation 701 may optionally include an output module 712 and a network interface 708. Memory 702 may store an application 718 and image data 77. Application 718 may include instructions executable by processor.

Application 718 may further include a user interface 716. Image data 714 may include the CT scans, the generated fluoroscopic 3D reconstructions of the target area and/or any other fluoroscopic image data and/or the generated one or more slices of the 3D reconstruction. Processor 704 may be coupled with memory 702, display 706, input device 710, output module 712, network interface 708 and imaging device 715. Workstation 701 may be a stationary computing device, such as a personal computer, or a portable computing device such as a tablet computer. Workstation 701 may embed a plurality of computer devices.

Memory 702 may include any non-transitory computer-readable storage media for storing data and/or software including instructions that are executable by processor 704 and which control the operation of workstation 701 and, in some embodiments, may also control the operation of imaging device 715. Imaging device 715 may be used to capture a sequence of fluoroscopic images based on which the fluoroscopic 3D reconstruction is generated and to capture a live 2D fluoroscopic view according to this disclosure. In an embodiment, memory 702 may include one or more storage devices such as solid-state storage devices, e.g., flash memory chips. Alternatively, or in addition to the one or more solid-state storage devices, memory 702 may include one or more mass storage devices connected to the processor 704 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 704. That is, computer readable storage media may include non-transitory, volatile, and non-volatile, removable and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by workstation 1001.

Application 718 may, when executed by processor 704, cause display 706 to present user interface 716. User interface 716 may be configured to present to the user a single screen including a three-dimensional (3D) view of a 3D model of a target from the perspective of a tip of a medical device, a live two-dimensional (2D) fluoroscopic view showing the medical device, and a target mark, which corresponds to the 3D model of the target, overlaid on the live 2D fluoroscopic view. User interface 716 may be further configured to display the target mark in different colors depending on whether the medical device tip is aligned with the target in three dimensions.

Network interface 708 may be configured to connect to a network such as a local area network (LAN) consisting of a wired network and/or a wireless network, a wide area network (WAN), a wireless mobile network, a Bluetooth network, and/or the Internet. Network interface 708 may be used to connect between workstation 701 and imaging device 715. Network interface 708 may also be used to receive image data 714. Input device 710 may be any device by which a user may interact with workstation 701, such as, for example, a mouse, keyboard, foot pedal, touch screen, and/or voice interface. Output module 712 may include any connectivity port or bus, such as, for example, parallel ports, serial ports, universal serial busses (USB), or any other similar connectivity port known to those skilled in the art. From the foregoing and with reference to the various figures, those skilled in the art will appreciate that certain modifications can be made to the disclosure without departing from the scope of the disclosure.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments.

This disclosure is further described in connection with the following examples.

Example 1-A control system, including a controller, the controller having a display; and a handle detachably coupled to the display, the handle including an input device, a motor assembly, wherein the display is detachably coupled to the motor assembly, the motor assembly including a drive motor in communication with the handle; and a catheter drive detachably coupled to the motor assembly, wherein actuation of the drive motor effectuates movement of a catheter operatively coupled to the catheter drive.

Example 2—The control system according to example 1, wherein the display includes

a first coupler and the handle includes a second coupler, wherein the first coupler is selectively couplable to the second coupler.

Example 3—The control system according to any of examples 1 or 2, wherein the first coupler includes a first magnet and the second coupler includes a second magnet.

Example 4—The control system according any of examples 1-3, wherein the first coupler includes a first plurality of magnets and the second coupler includes a second plurality of magnets, wherein the first plurality of magnets and the second plurality of magnets are disposed in a zig-zag configuration.

Example 5—The control system according to any of examples 1-4, wherein the first coupler includes a first electrical connector and the second coupler includes a second electrical connector, wherein the first electrical connector is selectively engageable with the second electrical connector.

Example 6—The control system according to example 5, wherein the handle includes an energy storage device, wherein the first electrical connector and the second electrical connector cooperate to supply electrical energy to the energy storage device.

Example 7—The control system according to example 1, further comprising a docking station detachably coupled to the controller.

Example 8—The control system according to example 7, wherein the docking station includes a first plurality of magnets and the display of the controller includes a second plurality of magnets, wherein the first plurality of magnets and the second plurality of magnets cooperate to selectively couple the controller to the docking station.

Example 9—The control system according to example 1, further comprising an insertion mechanism in communication with the handle, wherein the insertion mechanism is operably coupled to the catheter to effectuate selective movement of the catheter.

Example 10—The control system according to example 9, wherein the motor assembly and the catheter drive cooperate to effectuate roll and articulation of the catheter and the insertion mechanism effectuates insertion and retraction of the catheter, or wherein the insertion mechanism includes a control knob, the control knob operably coupled to the catheter, wherein rotation of the control knob in a first direction effectuates advancement of the catheter and rotation of the control knob in a second direction effectuates retraction of the catheter.

Example 11—A control station, including a controller having a housing, a first handle detachably coupled to the housing, a second handle detachably coupled to the housing; a motor assembly detachably coupled to the controller, the motor assembly including a drive motor in communication with each of the first handle and the second handle, and a catheter drive detachably coupled to the motor assembly, wherein an input received by the first handle effectuates articulation of a catheter operatively connected to the catheter drive and an input received by the second handle effectuates linear movement of the catheter.

Example 12—The control station according to example 11, wherein the motor assembly includes, a first drive motor operably coupled to a first output hub, a second drive motor operably coupled to a second output hub, a third drive motor operably coupled to a third hub; and a fourth drive motor operably coupled to a fourth hub.

Example 13—The control station according to example 12, wherein the catheter drive includes a first drive hub selectively engageable with the first output hub, a second drive hub selectively engageable with the second output hub, a third drive hub selectively engageable with the third output hub, and a fourth drive hub selectively engageable with the third output hub.

Example 14—The control station according to any of examples 11-13, wherein each of the first handle and the second handle are in wireless communication with the motor assembly.

Example 15—The control station according to any of examples 11-14, wherein the motor assembly is in wireless communication with the housing.

Example 16-A controller, including a housing having a display and a first plurality of magnetic elements, a handle detachably coupled to the housing, the handle in wireless communication with the housing, and a support including a second plurality of magnetic elements, wherein the second plurality of magnetic elements are selectively engageable with the first plurality of magnetic elements to selectively couple the housing to the support.

Example 17—The controller according to example 16, wherein the handle includes an energy storage device.

Example 18—The controller according to example 17, wherein the housing includes a first electrical connector and the handle includes a second electrical connector selectively engageable with the first electrical connector, the first electrical connector and the second electrical connector cooperating to transmit electrical energy from the housing to the energy storage device of the handle.

Example 19—The controller according to any of examples 16-18, wherein the housing includes a first magnetic coupling and the handle includes a second magnetic coupling selectively engageable with the first magnetic coupling to selectively couple the handle to the housing.

Example 20—The controller according to claim 19, wherein the first magnetic coupling includes a first plurality of magnetic elements disposed in a zig-zag configuration and the second magnetic coupling includes a second plurality of magnetic elements disposed in a complimentary configuration to the first plurality of magnetic elements.

What is claimed is:

1. A control system, comprising:
a controller, the controller including:
a display; and
a handle detachably coupled to the display, the handle including an input device;
a motor assembly, wherein the display is detachably coupled to the motor assembly, the motor assembly including a drive motor in communication with the handle; and
a catheter drive detachably coupled to the motor assembly, wherein actuation of the drive motor effectuates movement of a catheter operatively coupled to the catheter drive.

2. The control system according to claim 1, wherein the display includes a first coupler and the handle includes a second coupler, wherein the first coupler is selectively couplable to the second coupler.

3. The control system according to claim 2, wherein the first coupler includes a first magnet and the second coupler includes a second magnet.

4. The control system according to claim 2, wherein the first coupler includes a first plurality of magnets and the second coupler includes a second plurality of magnets, wherein the first plurality of magnets and the second plurality of magnets are disposed in a zig-zag configuration.

5. The control system according to claim 2, wherein the first coupler includes a first electrical connector and the second coupler includes a second electrical connector, wherein the first electrical connector is selectively engageable with the second electrical connector.

6. The control system according to claim 5, wherein the handle includes an energy storage device, wherein the first electrical connector and the second electrical connector cooperate to supply electrical energy to the energy storage device.

7. The control system according to claim 1, further comprising a docking station detachably coupled to the controller.

8. The control system according to claim 7, wherein the docking station includes a first plurality of magnets and the display of the controller includes a second plurality of magnets, wherein the first plurality of magnets and the second plurality of magnets cooperate to selectively couple the controller to the docking station.

9. The control system according to claim 1, further comprising an insertion mechanism in communication with the handle, wherein the insertion mechanism is operably coupled to the catheter to effectuate selective movement of the catheter.

10. The control system according to claim 9, wherein the motor assembly and the catheter drive cooperate to effectuate roll and articulation of the catheter and the insertion mechanism effectuates insertion and retraction of the catheter, or
wherein the insertion mechanism includes a control knob, the control knob operably coupled to the catheter, wherein rotation of the control knob in a first direction effectuates advancement of the catheter and rotation of the control knob in a second direction effectuates retraction of the catheter.

11. A control station, comprising:
a controller, the controller including:
a housing;
a first handle detachably coupled to the housing;
a second handle detachably coupled to the housing;
a motor assembly detachably coupled to the controller, the motor assembly including a drive motor in communication with each of the first handle and the second handle; and
a catheter drive detachably coupled to the motor assembly, wherein an input received by the first handle effectuates articulation of a catheter operatively connected to the catheter drive and an input received by the second handle effectuates linear movement of the catheter.

12. The control station according to claim 11, wherein the motor assembly includes:
a first drive motor operably coupled to a first output hub;
a second drive motor operably coupled to a second output hub;
a third drive motor operably coupled to a third hub; and
a fourth drive motor operably coupled to a fourth hub.

13. The control station according to claim 12, wherein the catheter drive includes:
a first drive hub selectively engageable with the first output hub;
a second drive hub selectively engageable with the second output hub;
a third drive hub selectively engageable with the third output hub; and
a fourth drive hub selectively engageable with the third output hub.

14. The control station according to claim 11, wherein each of the first handle and the second handle are in wireless communication with the motor assembly.

15. The control station according to claim 11, wherein the motor assembly is in wireless communication with the housing.

16. A controller, comprising:
a housing including:
a display; and
a first plurality of magnetic elements;
a handle detachably coupled to the housing, the handle in wireless communication with the housing; and
a support including a second plurality of magnetic elements, wherein the second plurality of magnetic elements are selectively engageable with the first plurality of magnetic elements to selectively couple the housing to the support.

17. The controller according to claim 16, wherein the handle includes an energy storage device.

18. The controller according to claim 17, wherein the housing includes a first electrical connector and the handle includes a second electrical connector selectively engageable with the first electrical connector, the first electrical connector and the second electrical connector cooperating to transmit electrical energy from the housing to the energy storage device of the handle.

19. The controller according to claim 16, wherein the housing includes a first magnetic coupling and the handle includes a second magnetic coupling selectively engageable with the first magnetic coupling to selectively couple the handle to the housing.

20. The controller according to claim 19, wherein the first magnetic coupling includes a first plurality of magnetic elements disposed in a zig-zag configuration and the second magnetic coupling includes a second plurality of magnetic elements disposed in a complimentary configuration to the first plurality of magnetic elements.
